(19) United States
(12) Patent Application Publication
(10) Pub. No.: US 2006/0138352 A1
(43) Pub. Date: Jun. 29, 2006

(54) Electron beam irradiation apparatus, electron beam irradiation method, and apparatus for and method of manufacturing disc-shape object

(75) Inventor: Kazushi Tanaka, CHUO-KU (JP)

Correspondence Address: DARBY & DARBY P.C., P. O. BOX 5257, NEW YORK, NY, 10150-5257, US

(73) Assignee: TDK CORPORATION, TOKYO (JP)

(21) Appl. No.: 10/528,511

(22) PCT Filed: Sep. 17, 2003
(86) PCT No.: PCT/JP03/11815

§ 371 Date: Mar. 18, 2005

(30) Foreign Application Priority Data
Sep. 19, 2002 (JP) 2002-274122

(51) Int. Cl. A61N 5/00

(52) U.S. Cl. 250/492.3

(57) ABSTRACT

An electron beam irradiation apparatus and method capable of easily curing at least part of a surface layer and/or a resin layer, each composed of materials that are hard to be cured by irradiation of ultraviolet rays, and capable of substantially uniformizing an integrated irradiation dose of electron beams over an entire irradiated surface. A disc-shaped object manufacturing apparatus and method capable of efficiently forming, on the disc-shaped object, at least part of a surface layer and/or a resin layer. An electron beam irradiation apparatus has a rotary driving unit for rotationally driving a disc-shaped object, a shield container for rotatably accommodating the disc-shaped object, and an electron beam irradiation unit provided in the shield container so that an irradiated surface on the surface of the disc-shaped object is irradiated with electron beams.

### TECHNICAL FIELD

The present invention relates to an electron beam irradiation apparatus and electron beam irradiation method for irradiating electron beams and to an apparatus for and a method of manufacturing a disc-shaped object.

### BACKGROUND ARTS

Optical discs such as a CD (Compact Disc), a DVD (Digital versatile Disc), etc. have hitherto been utilized as optical information recording mediums. Over the recent years, however, there has been a progress of developing a blue semiconductor laser of which an oscillation wavelength is on the order of 400 nm. The development of a next generation high-density optical disc such as a high-density DVD, etc. capable of recording with a higher density than the general DVD, is conducted by use of this type of blue semiconductor laser.

FIG. 12 shows an example of a now-thinkable layer structure of this type of next generation high-density optical disc.

This high-density optical disc is structured such that a recording layer 91 for recording information, a light transmitting layer 92 that transmits laser beams for recording and reproducing so that the laser beams get incident on the recording layer 91 and a lubrication layer 93 taking contact with a member on the side of an optical pickup into consideration, are stacked in this sequence on a substrate 90 composed of a resin material such as polycarbonate, etc.

The light transmitting layer 92 and the lubrication layer 93 are, when formed, irradiated with ultraviolet rays after being coated for curing. When especially the lubrication layer, etc. is formed of a material such as silicone compound, fluorine compound, etc. that exhibit radical polymerization double-bond, however, there might be a case in which a characteristic as the lubrication layer deteriorates if a reaction initiator is added thereto. In such a case, if the reaction initiator is not added, the curing is hard to be done by the irradiation of the ultraviolet rays, and the lubrication layer exhibiting a sufficient quality can not be formed. (Refer to Japanese Patent Laid-Open Application Publication No. 4-019839, Japanese Patent Laid-Open Application Publication No. 11-162015, Japanese Patent Laid-Open Application Publication No. 7-292470, Japanese Patent Laid-Open Application Publication No. 2000-64042).

### DISCLOSURE OF THE INVENTION

It is an object of the present invention to provide, in view of the aforementioned problems inherent in the prior arts, an electron beam irradiation apparatus and an electron beam irradiation method capable of easily curing at least part of a surface layer and/or a resin layer such as a light transmitting layer, etc. thereunder, each composed of materials that are hard to be cured by irradiation of ultraviolet rays, and capable of substantially uniformizing an integrated irradiation dose of electron beams over an entire irradiated surface.

It is another object of the present invention to provide a disc-shaped object manufacturing apparatus and a disc-shaped object manufacturing method capable of capable of substantially uniformizing an integrated irradiation dose of electron beams over an entire irradiated surface and efficiently forming, on a disc-shaped object, at least part of a surface layer and/or a resin layer such as a light transmitting layer, etc. thereunder, each composed of materials that are hard to be cured by the irradiation of ultraviolet rays.

An electron beam irradiation apparatus according to the present invention comprises a rotary driving unit for rotationally driving a disc-shaped object, a shield container for rotatably accommodating the disc-shaped object, and an electron beam irradiation unit provided in the shield container so that a face to be irradiated on the surface of the disc-shaped object is irradiated with electron beams, is characterized in that when the irradiated surface to be irradiated is irradiated with the electron beams emitted from the electron beam irradiation unit during rotations of the disc-shaped object, an irradiation beam intensity of the electron beams is set larger on the side of an outer peripheral surface in a radial direction of the disc-shaped object than on the side of an inner peripheral surface.

According to the electron beam irradiation apparatus, the on-rotating disc-shaped object is irradiated with the electron beams and can be therefore efficiently irradiated with the electron beams having the larger energy than the ultraviolet rays have. It is therefore possible to easily cure at least part of the surface layer and/or the resin layer such as the light transmitting layer, etc. thereunder, composed of the materials that are hard to be cured by the irradiation of, e.g., the ultraviolet rays. Further, when irradiated with the electron beams, a linear speed is higher on the side of the outer periphery in the radial direction of the disc-shaped object than on the side of the inner periphery, and hence, corresponding to this correlation, an irradiation beam intensity of the electron beams can be set larger on the side of the outer peripheral surface than on the side of the inner peripheral surface, whereby the integrated irradiation dose of the electron beams is substantially uniformized over the entire irradiated surface of the disc-shaped object. Owing to this uniformization, it is possible to substantially uniformly and instantaneously cure at least part of the surface layer and/or the resin layer such as the light transmitting layer, etc. thereunder at a high efficiency, each composed of materials that are hard to be cured by irradiation of ultraviolet rays.

Note that the light transmitting layer involves using a resin as a main component and corresponds to the resin layer according to the present invention. The resin layer may also be multi-layered, wherein, e.g., a hard coat layer may be provided on the surface side of the layer composed mainly of the resin, and these layers are stacked to form the layer of which the main component is the resin. Further, the surface layer may be formed of a material, e.g., a lubricating layer forming material and a material exhibiting water repellency and oil repellency, which are different from the layer of which the main component is the resin. Moreover, such a layer may also be either single-layered or multi-layered. The lubricating layer is a layer of one mode included in a definition of the surface layer according to the present invention. In the following discussion, the terms “resin layer” and “lubricating layer” are employed as defined in the connotation given above.

In the electron beam irradiation apparatus, it is preferable that an acceleration voltage of the electron beam irradiation unit is 20 kV through 100 kV. With this contrivance, particularly, electron beam energy is efficiently applied to, e.g., the resin layer over a thin range from the surface, and the electron beams do not affect a substrate, etc. existing thereunder.

Further, it is preferable that the electron beam irradiation unit includes a plurality of electron beam irradiation tubes arranged in the radial direction. In this case, the plurality of electron beam irradiation tubes can be arranged substantially in the same direction in the radial direction, and may also be arranged substantially in a side-by-side relation as shown in, e.g., FIGS. 16 and 17 in different directions in the radial direction. In this instance, a definition of “substantially the same direction in the radial direction” represents a direction along the same straight line extending in the radial direction, and a definition of “different directions in the radial direction” represents directions along different straight lines extending differently in the radial direction. The “radial direction” herein connotes a direction extending radially from the center of rotation of the disc-shaped object and a direction extending toward the outer periphery of the disc-shaped object from a point eccentric from the center of rotation of the disc-shaped object.

Further, each of current values of the plurality of electron beam irradiation tubes is set so that the current value of the electron beam irradiation tube disposed on the side of the outer peripheral surface is larger than the current value of the electron beam irradiation tube disposed on the side of the inner peripheral surface, whereby the irradiation beam intensity of the electron beams can be set larger on the side of the outer peripheral surface on the face to be irradiated than on the side of the inner peripheral surface.

Moreover, the plurality of electron beam irradiation tubes respectively have irradiation windows through which the electron beams are irradiated toward the outside, and are arranged so that a distance from the face to be irradiated to the irradiation window is shorter in the electron beam irradiation tube on the side of the outer peripheral surface than a distance in the electron beam irradiation tube on the side of the inner peripheral surface, whereby the irradiation beam intensity of the electron beams is attenuated corresponding to the distance to the irradiated surface and can be therefore set larger on the side of the outer peripheral surface on the face than on the side of the inner peripheral surface.

Moreover, at least one of the plurality of electron beam irradiation tubes is disposed as shown in, e.g., FIG. 15 or 18 so that the irradiation window thereof is inclined close to the side of the outer peripheral surface of the face to be irradiated, whereby the irradiation beam intensity of the electron beams is attenuated corresponding to the distance to the face and can be therefore set larger on the side of the outer peripheral surface on the irradiated surface than on the side of the inner peripheral surface.

Furthermore, the electron beam irradiation unit includes an electron beam irradiation tube having an irradiation window through which the electron beams are irradiated to the outside, and the electron beam irradiation tube is disposed so that the irradiation window thereof is inclined close to the side of the outer peripheral surface of the face to be irradiated, whereby the irradiation beam intensity of the electron beams is attenuated corresponding to the distance to the face from the irradiation window having a fixed size even in the single electron beam irradiation tube and can be therefore set larger on the side of the outer peripheral surface on the face than on the side of the inner peripheral surface.

Moreover, it is preferable that an interior of the shield container is set in an atmosphere of an inert gas such a nitrogen gas, an argon gas, a mixture of these gasses, etc., and the shield container is provided with a gas introduction port and a gas discharge port through which the inert gas flows in the vicinity of the irradiation window. The irradiation window can be cooled off owing to a flow of this inert gas.

In this case, a temperature sensor is provided in the vicinity of the irradiation window, and a flow rate of the inert gas is adjusted based on a temperature measured by the temperature sensor, whereby the vicinity of the irradiation window can be controlled at a temperature equal to or lower than a fixed temperature.

Further, it is preferable that an oxygen concentration meter for measuring an oxygen concentration within the shield container, is provided. This oxygen concentration meter enables confirmation that the interior of the shield container is kept with a fixed or lower oxygen concentration. For example, an inhibition of radical reaction due to the oxygen in the vicinity of the irradiation surface of the disc-shaped object to be irradiated with the electron beams, is hard to occur, and preferable curing reaction can be ensured.

Moreover, it is preferable that a vacuumizing device for depressurizing the interior of the shield container is provided. This vacuumizing device enables the irradiation of the electron beams to be conducted within the shield container depressurized down to a predetermined pressure, and also enables the interior of the shield container to be easily efficiently replaced with the inert gas atmosphere.

Moreover, it is preferable that the shield container is openable/closable and composed of a metallic material such as steel, stainless steel, etc., and has a shield structure for shielding the electron beams emitted from the irradiation window. This structure makes it possible to shield the electron beams and secondary X-rays and to leak none of the electron beams and the secondary X-rays to the outside, therefore preferable in terms of taking a security measure against exposure. Note that an air-tightly closed structure for air-tightly closing the shield container be, it is preferable, provided in the vicinity of the shield structure. Owing to this contrivance, a material of an O-ring, etc. structuring the air-tightly closed structure is shielded from the electron beams and does not suffer material deterioration due to the irradiation of the electron beams.

Furthermore, the electron beam irradiation apparatus further comprises a shutter member disposed between the electron beam irradiation unit and the irradiated surface, and movable between an opening position for opening to permit transmission of the electron beams and a closing position for closing to block the electron beams, and a shutter driving mechanism for moving the shutter member so as to effect switchover to the irradiation and non-irradiation of the electron beams during rotations of the disc-shaped object. With this construction, it is possible to easily execute switchover control of the irradiation and the non-irradiation of the electron beams, and there is no necessity of ON/OFF-controlling a power source of the electron beam irradiation unit. Accordingly, a period of startup time of the electron beam irradiation unit is not required, and this is efficient when repeating the irradiation of the electron beams.

In this case, the shutter member is constructed to open and close at a higher speed than a peripheral speed on the outer periphery of the disc-shaped object. With this construction, it is possible to ignore a difference in irradiation time when opening and closing the shutter member. Note that the “irradiation time” according to the present invention indicates a period of time for which the disc-shaped object remains actually irradiated with the electron beams as described above.

An electron beam irradiation method according to the present invention is characterized by comprising a step of rotationally driving a disc-shaped object, and a step of irradiating a face to be irradiated of the on-rotating disc-shaped object with the electron beams emitted from an electron beam irradiation unit so that an irradiation beam intensity of the electron beams is set larger on the side of an outer peripheral surface in a radial direction of the disc-shaped object than on the side of an inner peripheral surface.

According to the electron beam irradiation method, the on-rotating disc-shaped object is irradiated with the electron beams and can be therefore efficiently irradiated with the electron beams having the larger energy than the ultraviolet rays have. It is therefore possible to easily cure the resin layer composed of a resin material that is hard to be cured by the irradiation of, e.g., the ultraviolet rays. Further, when irradiated with the electron beams, the linear speed is higher on the side of the outer periphery in the radial direction of the disc-shaped object than on the side of the inner periphery, and hence, corresponding to this correlation, the irradiation beam intensity of the electron beams can be set larger on the side of the outer peripheral surface than on the side of the inner peripheral surface, whereby the integrated irradiation dose of the electron beams is substantially uniformized over the entire irradiated surface of the disc-shaped object. Owing to this uniformization, it is possible to substantially uniformly and instantaneously cure the entire surface of, e.g., the resin layer at the high efficiency.

In the electron beam irradiation method, it is preferable that an acceleration voltage of the electron beam irradiation unit is 20 kV through 100 kV. With this contrivance, particularly, the electron beam energy is efficiently applied to, e.g., the resin layer over the thin range from the surface, and the electron beams do not affect a substrate, etc. existing thereunder.

Further, each of current values of a plurality of electron beam irradiation tubes arranged in the radial direction serving as the electron beam irradiation unit is set so that the current value of the electron beam irradiation tube disposed on the side of the outer peripheral surface is larger than the current value of the electron beam irradiation tube disposed on the side of the inner peripheral surface, whereby the irradiation beam intensity of the electron beams can be set larger on the side of the outer peripheral surface on the face than on the side of the inner peripheral surface.

Moreover, a distance from the irradiated surface to each of the electron beam irradiation windows of the plurality of electron beam irradiation tubes arranged in the radial direction serving as the electron beam irradiation is set so that the distance in the electron beam irradiation tube on the side of the outer peripheral surface is shorter than the distance in the electron beam irradiation tube on the side of the inner peripheral surface, whereby the irradiation beam intensity of the electron beams is attenuated corresponding to the distance to the face and can be therefore set larger on the side of the outer peripheral surface on the irradiated surface than on the side of the inner peripheral surface.

Further, at least one of the plurality of electron beam irradiation tubes is inclined so that the irradiation window thereof gets close to the side of the outer peripheral surface of the face to be irradiated, whereby the irradiation beam intensity of the electron beams is attenuated corresponding to the distance to the face from the irradiation window having a fixed size and can be therefore set larger on the side of the outer peripheral surface on the face than on the side of the inner peripheral surface.

Moreover, the electron beam irradiation tubes arranged as the electron beam irradiation unit are inclined so that the irradiation windows thereof get close to the side of the outer peripheral surface of the irradiated surface, whereby the irradiation beam intensity of the electron beams is attenuated corresponding to the distance to the irradiated surface from the irradiation window having a fixed size even in the single electron beam irradiation tube and can be therefore set larger on the side of the outer peripheral surface on the irradiated surface than on the side of the inner peripheral surface.

Furthermore, an air-tightly closable shield container rotatably accommodates the disc-shaped object, and an interior of the shield container is replaced with an inert gas atmosphere by introducing an inert gas into the interior of the shield container, whereby the interior of the shield container can be easily efficiently replaced with the inert gas atmosphere. Note that the inert gas be, it is preferable, introduced while measuring an oxygen concentration within the shield container.

Further, it is preferable that the inert gas is flowed through the vicinity of the irradiation window of the electron beam irradiation unit toward a gas discharge port from a gas introduction port, thereby cooling off the irradiation window. Note that a cooling temperature be, it is preferable, controlled by adjusting a flow rate of the inert gas on the basis of a temperature measured by a temperature sensor provided in the vicinity of the irradiation window.

An apparatus for manufacturing a disc-shaped object according to the present invention comprises the aforementioned electron beam irradiation apparatus, is characterized in that a resin layer and/or a surface layer formed on the disc-shaped object is cured by the irradiation of the electron beams.

According to the disc-shaped object manufacturing apparatus, the on-rotating disc-shaped object is irradiated with the electron beams and can be therefore efficiently irradiated with the electron beams having the larger energy than the ultraviolet rays have. It is therefore possible to easily cure a lubricating layer and the resin layer composed of the materials that are hard to be cured by the irradiation of the ultraviolet rays, and to efficiently form the lubricating layer/the resin layer on the disc-shaped object. Further, when irradiated with the electron beams, the linear speed is higher on the side of the outer periphery in the radial direction of the on-rotating disc-shaped object than on the side of the inner periphery, and hence, corresponding to this correlation, the irradiation beam intensity of the electron beams can be set larger on the side of the outer peripheral surface than on the side of the inner peripheral surface, whereby the integrated irradiation dose of the electron beams is substantially uniformized over the entire irradiated surface of the disc-shaped object. Owing to this uniformization, it is possible to substantially uniformly and instantaneously cure the resin layer, etc. over the entire surface at the high efficiency, and to improve a quality and productivity of the disc-shaped object.

A method of manufacturing a disc-shaped object according to the present invention involves the use of the aforementioned electron beam irradiation apparatus or the electron beam irradiation method described above, is characterized in that a resin layer and/or a surface layer formed on the disc-shaped object is cured by the irradiation of the electron beams.

According to the disc-shaped object manufacturing method, the on-rotating disc-shaped object is irradiated with the electron beams and can be therefore efficiently irradiated with the electron beams having the larger energy than the ultraviolet rays have. It is therefore possible to easily cure a lubricating layer/the resin layer composed of the materials that are hard to be cured by the irradiation of the ultraviolet rays, and to efficiently form the lubricating layer/the resin layer on the disc-shaped object. Further, when irradiated with the electron beams, the linear speed is higher on the side of the outer periphery in the radial direction of the on-rotating disc-shaped object than on the side of the inner periphery, and hence, corresponding to this correlation, the irradiation beam intensity of the electron beams can be set larger on the side of the outer peripheral surface than on the side of the inner peripheral surface, whereby the integrated irradiation dose of the electron beams is substantially uniformized over the entire irradiated surface of the disc-shaped object. Owing to this uniformization, it is possible to substantially uniformly and instantaneously cure the resin layer, etc. over the entire surface at the high efficiency, and to improve a quality and productivity of the disc-shaped object.

Moreover, in the disc-shaped object manufacturing method described above, the acceleration voltage is 20 kV through 100 kV, and therefore the electron beam energy is efficiently applied to the resin layer, etc. over the thin range from the surface, and the electron beams do not affect a substrate, etc. existing thereunder.

It should be noted that the disc-shaped object manufacturing method comprises, it is preferable, a step of forming a light transmitting layer on the pre-irradiation disc-shaped object, which is executed before the electron beam irradiation step. It is also preferable that the disc-shaped object manufacturing method further comprises a step of forming a lubricating layer on the light transmitting layer, wherein the light transmitting layer and the lubricating layer can be cured and bridged by the irradiation of the electron beams.

### BRIEF DESCRIPTION OF THE DRAWINGS

FIG. 1 is a side sectional view schematically showing an electron beam irradiation apparatus in a first embodiment;

FIG. 2 is a plan view schematically showing a shutter member and a shutter driving mechanism of the electron beam irradiation apparatus in FIG. 1;

FIG. 3 is a block diagram showing a control system of the electron beam irradiation apparatus in FIG. 1;

FIG. 4 is a flowchart showing an operation of the electron beam irradiation apparatus in FIG. 1;

FIG. 5 is a side sectional view schematically showing an apparatus for manufacturing a disc-shaped medium according to a second embodiment, and is also an explanatory view showing a process just anterior to the electron beam irradiation for forming a resin layer on the disc-shaped medium;

FIG. 6 is a side sectional view similar to FIG. 5, and is also an explanatory view showing the electron beam irradiation for forming the resin layer on the disc-shaped medium and a process of exchanging the disc-shaped medium with an external disc-shaped medium;

FIG. 7 is a side sectional view similar to FIG. 5, and is also an explanatory view showing the electron beam irradiation for forming the resin layer on the disc-shaped medium and a process of exchanging the disc-shaped medium with the external disc-shaped medium;

FIG. 8 is a side sectional view similar to FIG. 5, and is also an explanatory view showing a preparatory process (such as depressurizing an interior of an exchange chamber, replacing a nitrogen gas, etc.) to the process of exchanging the disc-shaped medium internally in order to form the resin layer on the disc-shaped medium;

FIG. 9 is a side sectional view similar to FIG. 5, and is also an explanatory view showing the process of exchanging the disc-shaped medium internally in order to form the resin layer on the disc-shaped medium;

FIG. 10 is an enlarged sectional view showing a shield member 55 in the manufacturing apparatus in FIGS. 5 through 9;

FIG. 11 is a flowchart showing respective steps of irradiating a disc-shaped medium with electron beams and respective steps of ejecting and supplying the disc-shaped medium in the manufacturing apparatus in FIGS. 5 through 9;

FIG. 12 is a diagram showing an example of a layer structure of an optical disc that can be manufactured by the manufacturing apparatus in FIGS. 5 through 9;

FIG. 13 is a graph schematically showing a distribution, on a face 2b, of an irradiation beam intensity of the electron beams emitted from an electron beam irradiation unit 11 constructed of electron beam irradiation tubes 31, 32 in FIGS. 1 and 2;

FIG. 14 is a view schematically showing an example of a configuration of changing relative positions, in an irradiating direction of the electron beams, of the electron beam irradiation tubes 31, 32 of the electron beam irradiation unit in FIGS. 1 and 2;

FIG. 15 is a side view showing an example of the configuration in which the electron beam irradiation tube of the electron beam irradiation unit in FIGS. 1 and 2 is inclined to the disc-shaped object;

FIG. 16 is a plan view showing a modified example in FIG. 2, wherein the two electron beam irradiation tubes 31, 32 are arranged in different radial directions on a disc-shaped object 2;

FIG. 17 is a plan view showing another modified example in FIG. 2, wherein three electron beam irradiation tubes 31, 32, 33 are arranged in different radial directions on the disc-shaped object 2;

FIG. 18 is a side view showing a modified example of the inclined configuration in FIG. 15; and

FIG. 19 is a plan view showing a plane position of the irradiation window of the electron beam irradiation tube in FIG. 18 with respect to the disc-shaped object.

### BEST MODE FOR CARRYING OUT THE INVENTION

An electron beam irradiation apparatuses according to a first embodiment of the present invention and a disc-shaped medium manufacturing apparatus according to a second embodiment of the present invention, will hereinafter be described with reference to the drawings.

### First Embodiment

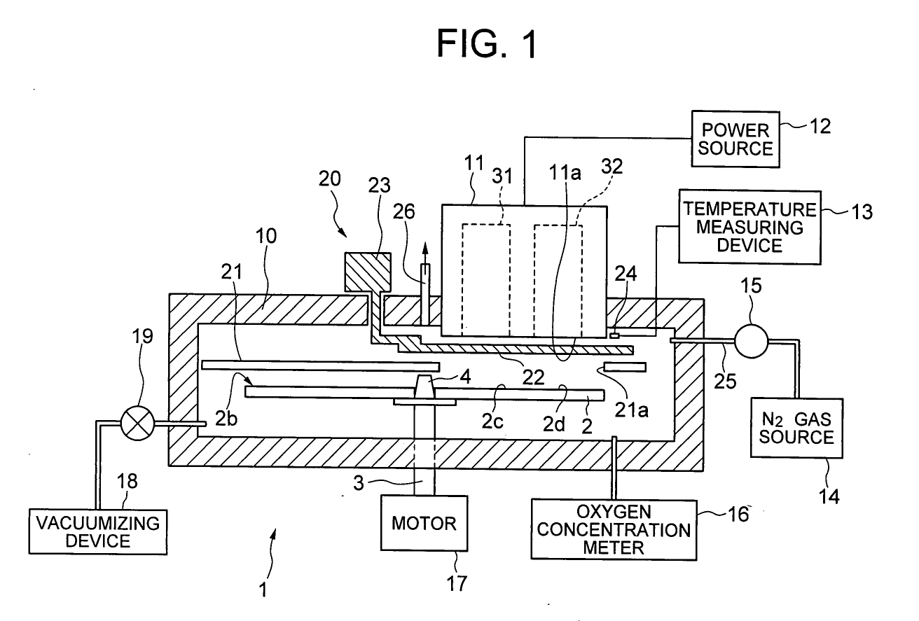

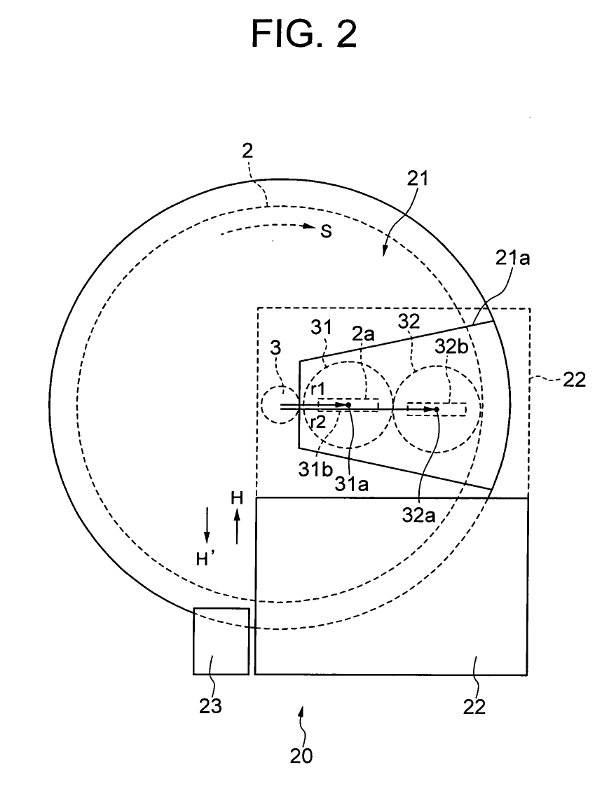

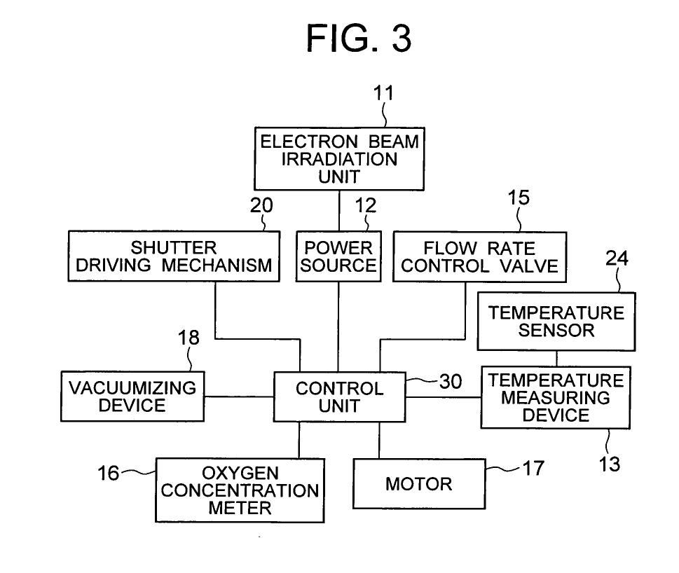

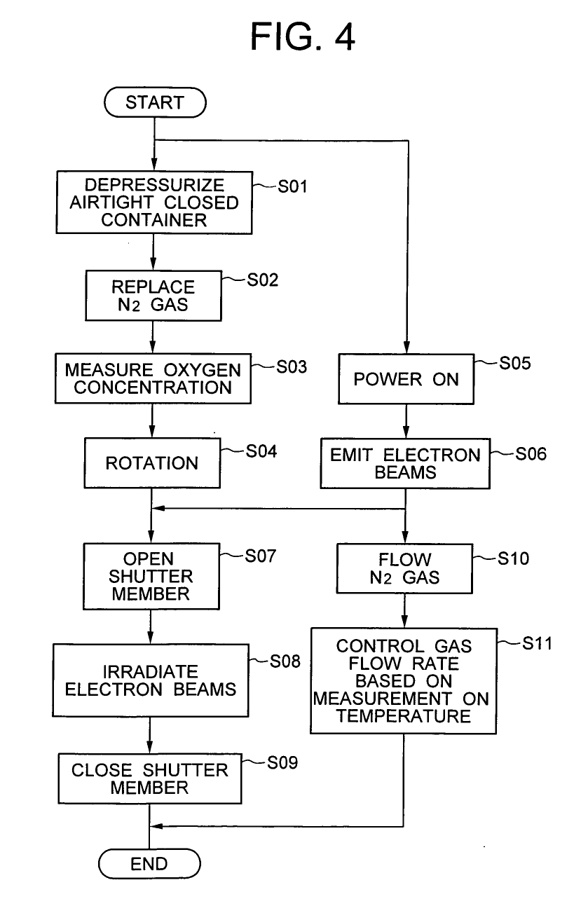

FIG. 1 is a side view schematically showing the electron beam irradiation apparatus in the embodiment of the present invention. FIG. 2 is a plan view schematically showing a shutter member and a shutter driving mechanism of the electron beam irradiation apparatus in FIG. 1. FIG. 3 is a block diagram showing a control system of the electron beam irradiation apparatus in FIG. 1. FIG. 4 is a flowchart showing an operation of the electron beam irradiation apparatus in FIG. 1.

As illustrated in FIG. 1, an electron beam irradiation apparatus 1 includes a shield container 10 that rotatably accommodates a disc-shaped object 2 and is composed of stainless steel in order to shield the electron beams, a motor 17 for rotationally driving the disc-shaped object 2 held by engaging a central hole of the disc-shaped object 2 with an engaging member 4 through a rotary shaft 3, an electron beam irradiation unit 11 for irradiating the disc-shaped object 2 with the electron beams in a radial direction from an irradiation surface 11a, a power source 12 for applying a voltage and flowing an electric current to the electron beam irradiation unit 11, a temperature sensor 24 disposed in the vicinity of the irradiation surface 11a, and a temperature measuring device 13 that is connected to the temperature sensor 24 and measures an ambient temperature to the irradiation surface 11a.

The electron beam irradiation apparatus 1 further includes an oxygen concentration meter 16 for measuring an oxygen concentration of oxygen in an airtight closed space within the shield container 10, a vacuumizing device 18 for evacuating and thus depressurizing an interior of the shield container 10 via a valve 19, a nitrogen gas source 14 that supplies a nitrogen gas for replacing the interior of the shield container 10 with a nitrogen gas atmosphere, and a gas flow rate control valve 15 capable of controlling a gas flow rate when the nitrogen gas flows so that the nitrogen gas is introduced via a gas introduction port 25, passes through in the vicinity of the irradiation surface 11a and is discharged from a gas discharge port 26. Further, the gas discharge port 26 is provided with a valve (unillustrated).

The electron beam irradiation apparatus 1 further includes an aperture-formed disc 21 having a larger diameter than that of the disc-shaped object 2 and disposed between the disc-shaped object 2 and the irradiation surface 11a of the electron beam irradiation unit 11, and a shutter driving mechanism 20 having a shutter member 22 disposed between the disc 21 and the irradiation surface 11a, and a slider 23 for driving the shutter member 22.

As shown in FIG. 2, the disc 21 has a fan-shaped aperture 21a, wherein the electron beams emitted from the electron beam irradiation unit 11 pass through this aperture 21a and fall upon a radial area 2a formed between an inner peripheral side portion and an outer peripheral side portion of the disc-shaped object 2 in the radial direction. Then, the disc-shaped object 2 is rotated, and hence an entire surface of a face 2b to be irradiated (FIG. 1), of the disc-shaped object 2 is irradiated with the electron beams.

Further, the shutter member 22 is formed in a rectangular shape and, when driven by the slider 23 in a slide direction H in FIG. 2, as depicted by a broken line in FIG. 2, moves to a closing position for completely covering and closing the fan-shaped aperture 21a of the disc 21 and thus blocks the electron beams emitted from the electron beam irradiation unit 11, with the result that the radial area 2a of the disc-shaped object 2 is not irradiated with the electron beams. Further, the shutter member 22, when driven by the slider 23 in a slide direction H′ opposite to the direction H, as depicted by a solid line in FIG. 2, moves completely off the aperture 21a back to an opening position for opening the aperture 21a, whereby the electron beams emitted from the electron beam irradiation unit 11 are permitted to pass therethrough and fall upon the radial area 2a of the disc-shaped object 2.

Further, as illustrated in FIGS. 1 and 2, the electron beam irradiation unit 11 includes a plurality of cylindrical electron beam irradiation tubes 31, 32 arranged on the side of an inner peripheral surface 2c and on the side of an outer peripheral surface 2d, in the radial direction, of the disc-shaped object 2. A voltage is applied to each of the electron beam irradiation tubes 31, 32 from the power source 12, and the electron beams, of which an acceleration voltage is on the order of 20 kV through 100 kV, travel through the irradiation windows 31b, 32b and fall upon the radial area 2a of the disc-shaped object 2.

Next, effects yielded by changing the tube currents to the respective electron beam irradiation tubes 31, 32 will be explained.

Supposing that t sec is set as a period of time required for one rotation when the rotated body 1 is rotated at a fixed speed in a rotating direction S at the irradiation time of the electron beams in FIG. 2, a peripheral speed v1 in a radial position r1 of the rotated body 2 and a peripheral speed v2 in a radial position r2 thereof can be expressed in the following formulae, respectively.
v1=(2π·r1)/t (1)
v2=(2π·r2)/t (2)
where r1<r2, and hence a relation between the peripheral speed v1 and the peripheral speed v2 is given in the following formula (3).
v1<v2 (3)

Note that the electron beam irradiation tubes 31, 32 are arranged so that a center 31a of the electron beam irradiation tube 31 is coincident with the radial position r1 of the disc-shaped object 2, and that a center 32a of the electron beam irradiation tube 32 is coincident with the radial position r2.

As described above, in the disc-shaped object 2 rotated at the fixed rotating speed, the peripheral speed differs depending on the radial position on the surface of the disc-shaped object 2 as shown in the formula (3), and therefore an integrated irradiation dose of the electron beams has an ununiform distribution, wherein the dose is large on the side

The respective irradiation windows 31b, 32b are, as depicted by broken lines in FIG. 2, each constructed in an elongate rectangular shape and are arranged extending in the radial direction of the disc-shaped object 2. The irradiation windows 31b, 32b are flush with (on the same plane as) the irradiation surface 11a of the electron beam irradiation unit 11 as shown in FIG. 1.

Further, the power source 12 is capable of changing tube currents flowing to the respective electron irradiation tubes 31, 32, wherein the tube current flowing to the electron beam irradiation tube 32 disposed on the side of the outer peripheral surface 2d of the disc-shaped object 2, is set larger than the tube current to the electron beam irradiation tube 31 disposed on the side of the inner peripheral surface 2c.

FIG. 13 is a schematic graph of an irradiation beam intensity distribution of the electron beams emitted from the electron beam irradiation unit 11 constructed of the electron beam irradiation tubes 31, 32 described above. As understood from FIG. 13, the irradiation beam intensity of the electron beams can be set larger on the side of the outer peripheral surface 2d on the irradiation surface 2b of the disc-shaped object 2 than on the side of the inner peripheral surface 2c by changing the tube current in the way described above.

In this case, the tube currents to the electron beam irradiation tubes 31, 32 can be set to, e.g., 300 μA, 600 μA, respectively.

of the inner peripheral surface 2c but small on the side of the outer peripheral surface 2d in the radial area 2a. However, the irradiation beam intensity of the electron beams can be set comparatively larger on the side of the outer peripheral surface 2d and set comparatively small on the side of the inner peripheral surface 2c as shown in FIG. 13 by changing the tube currents to the electron beam irradiation tubes 31, 32 as described above, thereby making it possible to correct and comparatively uniformize the ununiform distribution of the integrated irradiation dose of the electron beams in the radial direction.

Note that a moving speed when the shutter driving mechanism 20 opens and closes the shutter member 22 with the aid of the slider 23 is comparatively high and is by far higher than the peripheral speed on the outer periphery of the disc-shaped object, and hence there can be ignored a time difference of the irradiation that occurs when opening and closing the shutter member.

The thus-constructed electron beam irradiation apparatus 1 in FIGS. 1 and 2, irradiates the electron beams in a way that controls the whole as shown in FIG. 3 by the control unit 30, and respective steps S01 through S11 of the operation of the electron beam irradiation apparatus 1 will be described with reference to FIG. 4.

Under the control of the control unit 30, to begin with, after closing the valve at the gas discharge port 26, the vacuumizing device 18 operates to depressurize the interior of the shield container 10 (S01), then the valve 19 is closed, and the nitrogen gas is introduced into the shield container 10 via a gas flow rate control valve 15 from the nitrogen gas source 14 (S02). The interior of the shield container 10 can be thereby easily replaced with a nitrogen atmosphere.

Then, the oxygen concentration meter 16 detects a decrease down to a predetermined oxygen concentration in the interior of the shield container 10 (S03), and the disc-shaped object 2 is rotated at a predetermined rotating speed by driving the motor 17 (S04). On the other hand, the voltage is applied to the electron beam irradiation unit 11 from the power source 12 (S05), thereby generating the electron beams (S06). At this time, the shutter member 22 is in the closing position, thereby controlling an emission quantity of the electron beams down to a small level.

Next, the shutter member 22 existing in the closing position shown by the broken line in FIG. 2 is moved in the slide direction H′ to the opening position by operating the shutter driving mechanism 20 and thus driving the slider 23, thereby unclosing the aperture 21a (S07). At the same time, the emission quantity of the electron beams is controlled up to a large level, and the surface of the radial area 2a of the on-rotating disc-shaped object 2 is irradiated with the electron beams (S08). Thus, the radial area 2a of the on-rotating disc-shaped object 2 is irradiated with the electron beams, and hence the whole surface of the disc-shaped object 2 can be irradiated with the electron beams.

Then, after irradiating the disc-shaped object 2 with the electron beams for only a predetermined period of time, similarly the shutter member 22 is moved in the slide direction H by operating the shutter driving mechanism 20 to the closing position, thereby closing the aperture 21a (S09). Then, the irradiation of the electron beams upon the disc-shaped object 2 is finished.

Further, during the emission of the electron beams from the electron beam irradiation unit 11, the nitrogen gas from the nitrogen gas source 14 flows through the vicinity of the irradiation surface 11a via the gas introduction portion 25 and further flows into the gas discharge portion 26 (S10), thereby making it possible to cool off the irradiation surface 11a that rises in its temperature when emitting the electron beams and likewise cool off the shutter member 22. Moreover, a temperature ambient to the irradiation surface 11a is measured by the temperature sensor 24 and by the temperature measuring device 13, and a flow rate of the nitrogen gas is controlled based on this measured temperature by the gas flow rate control valve 15 (S11). The temperature ambient to the irradiation surface 11a can be controlled to be equal to or lower than a fixed temperature.

As described above, according to the electron beam irradiation apparatus in FIGS. 1 through 4, the surface of the on-rotating disc-shaped object 2 is irradiated with the electron beams, thereby enabling the surface of the disc-shaped object 2 to be highly efficiently irradiated with the electron beams exhibiting greater energy than the ultraviolet rays have. It is therefore feasible to facilitate curing of a resin layer, etc. made of a material that is hard to be cured by the irradiation of, for example, the ultraviolet rays.

Further, the surface of the disc-shaped object 2 is irradiated with the electron beams of which the acceleration voltage is 20 kV through 100 kV, whereby the electron beam energy can be highly efficiently applied across the surface of the disc-shaped object 2 over a thin range, e.g., over the resin layer, and deterioration of a substrate, etc. can be prevented without exerting influence of the electron beams upon the substrate, etc. existing thereunder.

Further, switchover control of the irradiation and non-irradiation of the electron beams can be easily executed by use of the shutter driving mechanism 20 and the shutter member 22.

Moreover, the irradiation of the electron beams can be effected to attain substantially the uniform distribution of the integrated irradiation dose of the electron beams in the radial direction of the disc-shaped object 2, and the energy of the electron beams can be applied uniformly to the whole of the face 2b of the disc-shaped object 2, whereby, e.g., the resin layer can be substantially uniformly and instantaneously cured on the entire face 2b at high efficiency.

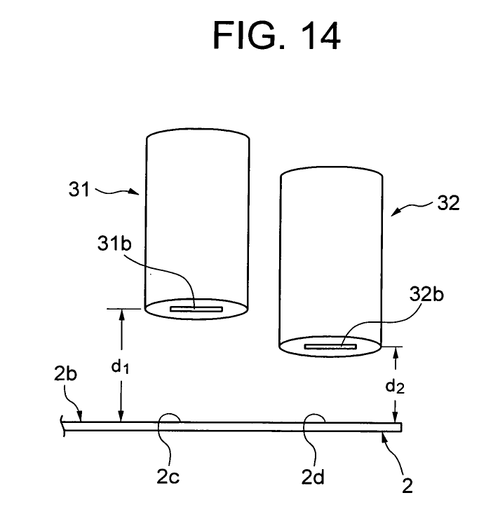

Next, a different example of the construction in which the irradiation beam intensity of the electron beams is set larger on the side of the outer peripheral surface 2d on the face 2b of the disc-shaped object than on the side of the inner peripheral surface 2c, will be explained with reference to FIG. 14.

FIG. 14 is a view schematically showing relative positions, in an irradiating direction of the electron beams, of the electron beam irradiation tubes 31, 32 of the electron beam irradiation unit in FIGS. 1 and 2.

As illustrated in FIG. 14, the electron beam irradiation tube 31 is disposed on the side of the inner peripheral surface 2c of the disc-shaped object 2, while the electron beam irradiation tube 32 is disposed on the side of the outer peripheral surface 2d of the disc-shaped object 2, wherein a distance d2 from an irradiation window 32b of the electron beam irradiation tube 32 to the face 2b is set shorter than a distance d1 from an irradiation window 31b of the electron beam irradiation tube 31 to the face 2b. The electron beams get more attenuated as the distance becomes longer, and hence, as in the case of FIG. 13, the irradiation beam intensity distribution of the electron beams can be set larger on the side of the outer peripheral surface 2d of the disc-shaped object 2 than on the side of the inner peripheral surface 2c.

As described above, in the disc-shaped object 2 rotated at the fixed rotating speed, the speed differs depending on the radial position on the surface of the disc-shaped object 2 as understood from the formula (3), so that there is plotted the ununiform distribution wherein the integrated irradiation dose of the electron beams becomes large on the side of the inner peripheral surface 2c but small on the side of the outer peripheral surface 2d. As explained above, however, the irradiation beam intensity of the electron beams can be set comparatively large on the side of the outer peripheral surface 2d and comparatively small on the side of the inner peripheral surface 2c by changing the distances of the electron beam irradiation tubes 31, 32 to the face 2b. It is therefore feasible to correct and comparatively uniformize the ununiform distribution in the radial direction of the integrated irradiation dose of the electron beams.

Accordingly, the irradiation of the electron beams can be conducted to attain substantially the uniform distribution of the integrated irradiation dose of the electron beams in the radial direction of the disc-shaped object 2, and the energy of the electron beams can be applied uniformly to the whole of the face 2b of the disc-shaped object 2, whereby, e.g., the resin layer can be substantially uniformly and instantaneously cured on the entire face 2b at the high efficiency.

Note that the distribution of the integrated irradiation dose of the electron beams in the radial direction can be so adjusted as to be more uniformized by changing the distances d1, d2 of the respective electron beam irradiation tubes 31, 32 in FIG. 14. Moreover, the tube currents and the acceleration voltages of the electron beam irradiation tubes 31, 32 may be set to the same values in FIG. 14, however, at least one of the tube current and the acceleration voltage may also be changed in the way explained above.

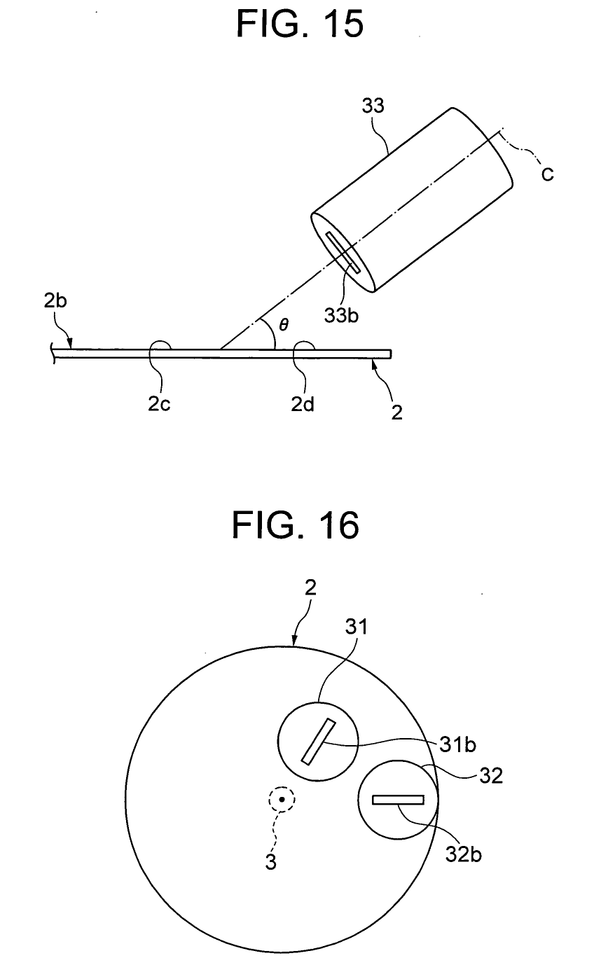

Next, a still different example of the construction in which the irradiation beam intensity of the electron beams is set larger on the side of the outer peripheral surface 2d on the face 2b of the disc-shaped object than on the side of the inner peripheral surface 2c, will be explained with reference to FIG. 15.

FIG. 15 is a view schematically showing a state where the electron beam irradiation tube of the electron beam irradiation unit in FIGS. 1 and 2 is inclined to the disc-shaped object.

As shown in FIG. 15, a single electron beam irradiation tube 33, which is the same as the electron beam irradiation tubes 31, 32, is disposed obliquely so that its irradiation window 33b gets proximal to the side of the outer peripheral surface 2d of the face 2b of the disc-shaped object 2. With this configuration, the irradiation beam intensity of the electron beams is attenuated corresponding to a distance from the irradiation window 33b having a fixed size to the irradiated surface, and can be therefore set larger on the side of the outer peripheral surface 2d of the irradiated surface than on the side of the inner peripheral surface 2c. Accordingly, in the same way as described above, it is possible to correct and to comparatively uniformize the ununiform distribution of the integrated irradiation beam intensity of the electron beams in the radial direction. In this case, the distribution of the integrated irradiation dose of the electron beams in the radial direction can be so adjusted as to be more substantially uniformized by properly changing an angle θ (an angle as viewed just from sideways as in FIG. 15) made by a longitudinal central axis c of the electron beam irradiation tube 33 and by the face 2b.

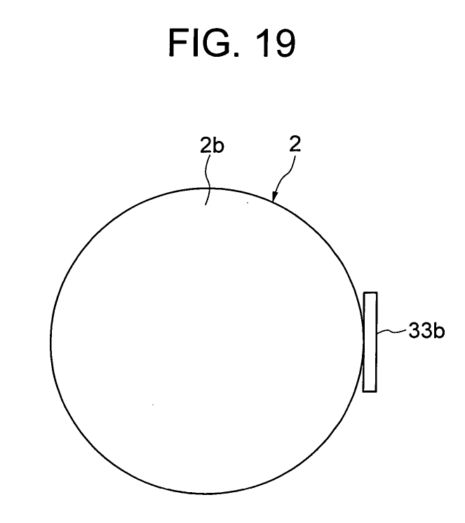

Further, in FIG. 15, the electron beam irradiation tube 33 is disposed so that a lower edge of the elongated rectangular irradiation window 33b is set closest to the face 2b of the disc-shaped object 2. As illustrated in FIGS. 18 and 19, however, the electron beam irradiation tube 33 may also be disposed so that a long side of the irradiation window 33b is set substantially parallel with the face 2b.

Note that the single electron beam irradiation tube is disposed in FIG. 15, however, a plurality of electron beam irradiation tubes are arranged in a way that similarly takes the inclined structure in which part or the whole of the electron beam irradiation tubes may be inclined. Further, a height from the disc-shaped object may also be changed. Moreover, the tube current of at least one of the electron beam irradiation tubes may be changed as described above.

### Second Embodiment

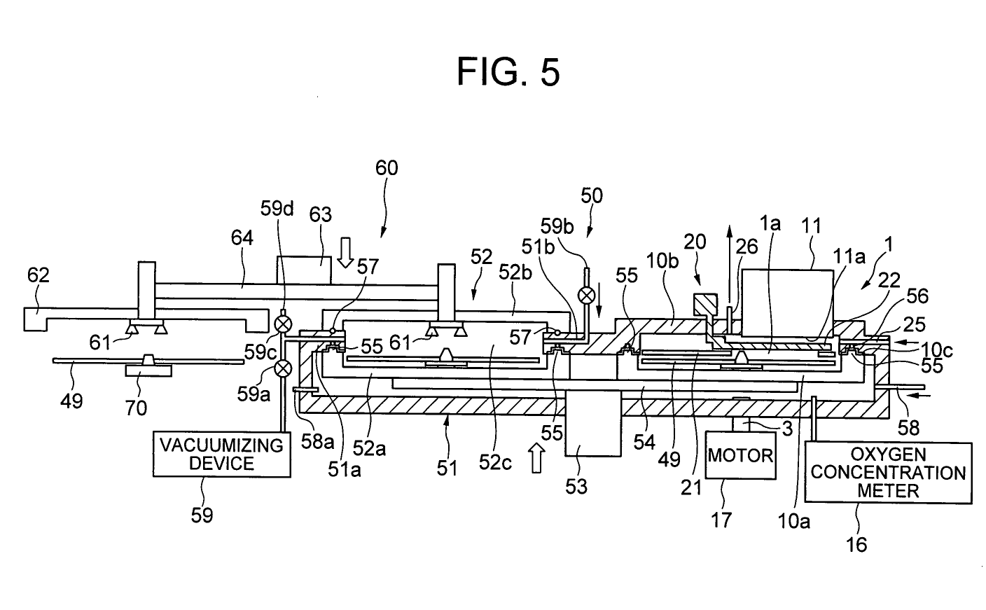

Next, an apparatus for manufacturing the disc-shaped medium will be described by way of a second embodiment. FIGS. 5 through 9 are side views of the manufacturing apparatus, explaining respective processes for forming the lubricating layer on the disc-shaped medium according to the second embodiment.

As shown in FIGS. 5 through 9, a disc-shaped medium manufacturing apparatus (which will hereinafter be simply termed a [manufacturing apparatus]) 50 has an airtight closable chamber 51 accommodating the electron beam irradiation apparatus 1 that emits the electron beams of which the acceleration voltage is as low as 20 kV through 100 kV and irradiates the surface of a disc-shaped medium 49 with the electron beams, an exchange chamber 52 for supplying (loading) the pre-irradiation disc-shaped medium 49 into the electron beam irradiation apparatus 1 and receiving a post-irradiation disc-shaped medium 49a from the electron beam irradiation apparatus 1, and a rotational (turn) unit 54 that rotates about a rotary shaft 53 in order to exchange the pre-irradiation disc-shaped medium with the post-irradiation disc-shaped medium.

As shown in FIGS. 5 through 9, the manufacturing apparatus 50 further includes a disc carrying device 60 for carrying the disc-shaped medium in a way that loads the pre-irradiation disc-shaped medium into the exchange chamber 52 and ejects the post-irradiation disc-shaped medium.

The electron irradiation apparatus 1 is constructed substantially in the same way as in FIGS. 1 and 2, and hence a different point from the configuration in FIGS. 1 and 2 will be explained. To be specific, the shield container 10 in FIG. 1 is, referring to FIG. 5, divided into a rotational (turn) tray unit 10a, provided on a lower side as viewed in FIG. 5, for rotatably accommodating the disc-shaped medium 49, and an upper-side fixed unit 10b provided with the electron beam irradiation unit 11, the shutter driving mechanism 20, etc. The rotational tray unit 10a is movable to the side of the exchange chamber 52 in a way that moves up and down and turns with the aid of the rotational unit 54 with respect to the fixed unit 10b.

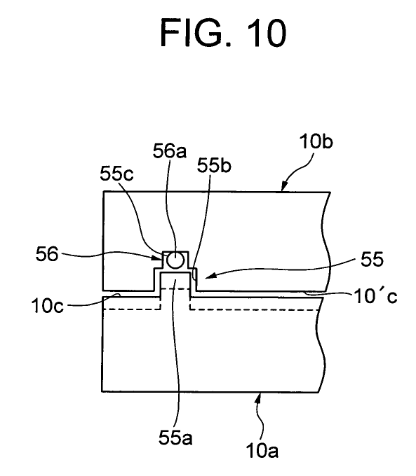

As illustrated in FIG. 5, an abutting face 10c of the rotational tray unit 10a and an abutting face 10c′ of the fixed unit 10b are provided with shield members 55 for shielding the electron beams so that the electron beams do not leak out. FIG. 10 is an enlarged sectional view showing the shield member 55. As shown in FIG. 10, the abutting face 10c of the rotational tray unit 10a has a protruded portion 55a formed along the entire periphery thereof, and the abutting face 10c′ of the fixed unit 10b has a recessed portion 55b formed along the entire periphery thereof, wherein the protruded portion 55a can be fitted in the recessed portion 55b.

Further, a bottom of the recessed portion 55b configuring the shield member 55 is further formed with a cavity 55c, and an O-ring 56a is accommodated in the cavity 55c, thus forming an airtight closed portion 56. The rotational tray unit 10a abuts on the fixed unit 10b, thereby making it possible to enhance airtightness in an airtight closed space 1a formed inside owing to the airtight closed portion 56.

In FIG. 10, the O-ring 56a in the airtight closed portion 56 is positioned much closer to the bottom within the cavity 55c from the recessed portion 55b and is not therefore irradiated with the electron beams directly, whereby the O-ring 56a can be prevented from being deteriorated.

As illustrated in FIG. 5, the exchange chamber 52 includes a rotational tray unit 52a that is moved up and down and rotated by the rotational unit 54 and is thus moved to the side of the electron beam irradiation apparatus 1, wherein this rotational tray unit 52a is exchangeable with the rotational tray unit 10a. The exchange chamber 52 further includes a carry rotational tray unit 52b that receives the pre-irradiation disc-shaped medium and ejects the post-irradiation disc-shaped medium to the outside by use of the disc carrying device 60.

The chamber 51 has an edge portion 51a and a connecting portion 51b that configure part of the exchange chamber 52. The edge portion 51a and the connecting portion 51b are interposed serving as abutting faces between the rotational tray unit 52a and the carry rotational tray unit 52b of the exchange chamber 52, whereby an airtight closed space 52c is formed within the exchange chamber 52 and at the same time the carry rotational tray unit 52b configures part of the chamber 51.

Moreover, airtight closed portions 57 each using an O-ring are provided on an abutting face between the edge portion 51a and the carry rotational tray unit 52b and on an abutting face between the edge portion (connecting portion) 51b and the carry rotational tray unit 52b. Further, the same shield portions 55 and the same airtight closed portions 56 as those in FIG. 10 are respectively provided on the abutting face between the edge portion 51a and the rotational tray unit 52a and on the abutting face between the connecting portion 51b and the rotational tray unit 52a.

The chamber 51 connects to the fixed unit 10b on the side of the edge portion of the electron beam irradiation apparatus 1, the connecting portion 51b connects to the fixed unit 10b in the vicinity of a central portion, and the carry rotational tray unit 52b is air-tightly closed by the edge portion 51a and by the connecting portion 51b, thereby becoming air-tightly closable on the whole. Moreover, the chamber 51, the carry rotational tray unit 52b (62), the rotational tray unit 10a, the fixed unit 10b, etc., are made of iron and steel, stainless steel and so on, thereby shielding the electron beams to prevent the electron beams from leaking to the outside.

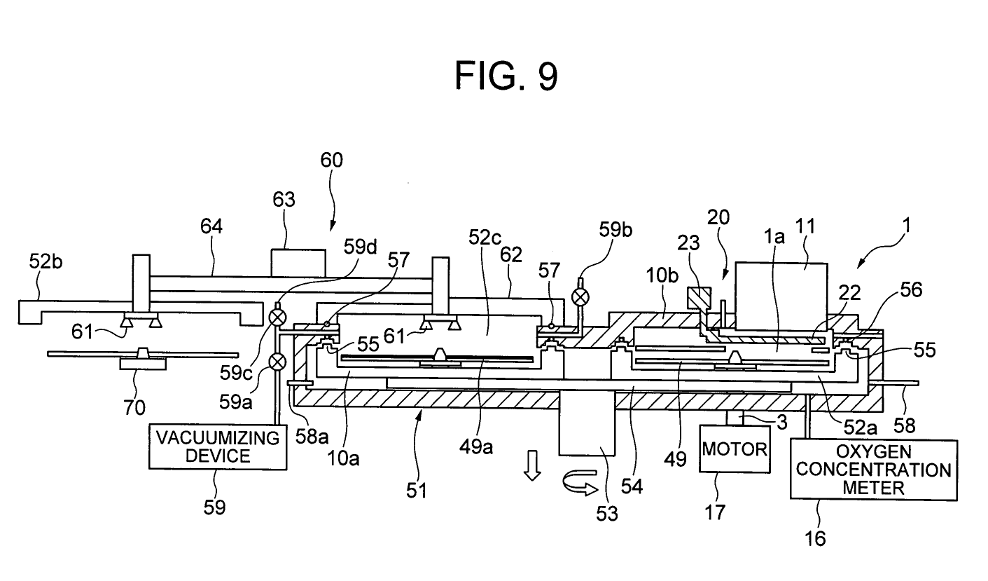

The nitrogen gas can be introduced into the chamber 51 via a nitrogen gas introduction port 58, and the airtight closed space 52c within the exchange chamber 52 can be depressurized by a vacuumizing device 59. As shown in FIG. 9, in a state where the whole chamber 51 is air-tightly closed, the rotational unit 54 moves together with the rotational tray units 10a, 52a downward as viewed in FIG. 9, and the airtight closed spaces 1a, 52c are opened. This case indicates a state in which (the interior of) the exchange chamber 52 is replaced with the nitrogen gas, and hence the interior of the chamber 51 does not affect the nitrogen gas atmosphere in the airtight closed space 1a of the electron beam irradiation apparatus 1.

Moreover, the nitrogen gas can be introduced into the exchange chamber 52 via a nitrogen gas introduction port 59b. Further, the nitrogen gas in the chamber 51 can be discharged from a gas discharge port 58a.

As shown in FIG. 5, the disc carrying device 60 includes another carry rotational tray unit 62 exchangeable with the carry rotational tray unit 52b configuring the exchange chamber 52, and a rotational unit (rotational plate) 64 that rotates the carry rotational tray units 52b, 62 through a rotary shaft 63. Each of the carry rotational tray units 52b, 62 has an adsorbing member 61 for vacuum-adsorbing the disc-shaped medium 49 in the vicinity of the periphery of a central hole of the disc-shaped medium 49. The rotational unit 64 makes the up-and-down and rotational movements and thus carries the disc-shaped medium between the exchange chamber 52 and an external disc transferring/receiving unit 70.

The disc-shaped medium 49 supplied from the disc transferring/receiving unit 70 to the exchange chamber 52 is formed on its surface with a light transmitting layer containing a resinous material and a lubricating layer composed of a lubricant thereon by use of an external spin coat device.

A material for forming this type of light transmitting layer is not particularly limited on condition that it is an active energy ray curing compound. It is, however, preferable that this material contains at least one reactive group selected from within a (meta) acryloyl group, a vinyl group and a mercapto group. For others, the aforementioned material may contain a known photo-polymerization initiator.

Further, for example, a silicone compound and a fluorine compound each exhibiting radical polymerization property are given as materials for forming the lubricating layer. The materials are not, however, limited to those aforementioned. Those lubricating layer forming materials are generally hard to be cured by ultraviolet rays in the case of containing no photo-polymerization initiator but can be instantaneously cured by the electron beams.

Next, an operation of the manufacturing apparatus 50 described above will be explained with reference to flowcharts in FIGS. 5 through 9 and 11 in a way that divides the operation into the irradiation of the electron beams upon the disc-shaped medium and the ejecting/supplying of the disc-shaped medium.

<Irradiation of Electron Beams upon Disc-Shaped Medium>

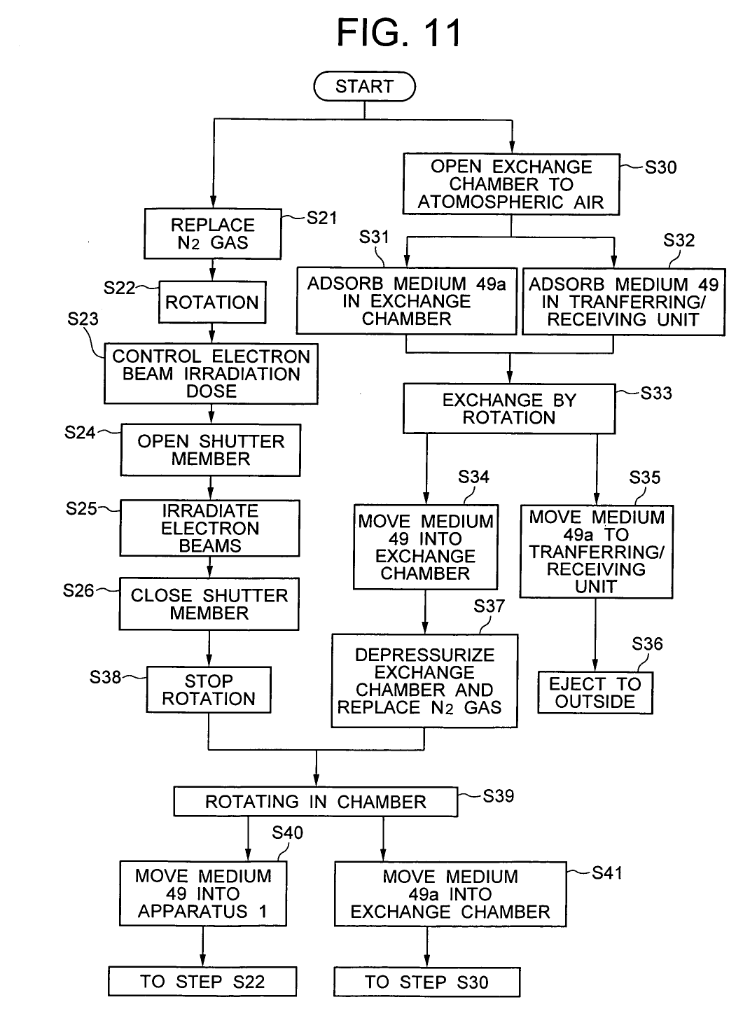

As shown in FIG. 11, to begin with, the whole chamber 51 is air-tightly closed as illustrated in FIG. 9, and the rotary shaft 53 and the rotational unit 54 moves downward as viewed in FIG. 9 together with the rotational tray units 10a, 52a. Then, after the airtight closed spaces 1a, 52c have been opened, the nitrogen gas is introduced into the chamber 51 via the nitrogen gas introduction port 58, thereby replacing the interior thereof with the nitrogen gas atmosphere (S21). At this time, the replacement with the nitrogen gas can be performed while measuring a concentration of oxygen in the chamber 51 by the oxygen concentration meter 16.

Next, when the rotary shaft 53 and the rotational unit 54 move upward as viewed in the Figure together with the rotational tray units 10a, 52a, as shown in FIG. 5, the airtight closed spaces 1a, 52c are formed. Then, in the electron beam irradiation apparatus 1, the disc-shaped medium 49 is rotated by the motor 17 within the airtight closed space 1a (S22), the electron beam irradiation unit 11 is controlled to emit a predetermined amount of electron beams (S23), and the nitrogen gas flows through the vicinity of the irradiation surface 11a toward the gas discharge port 26 from the gas introduction port 25.

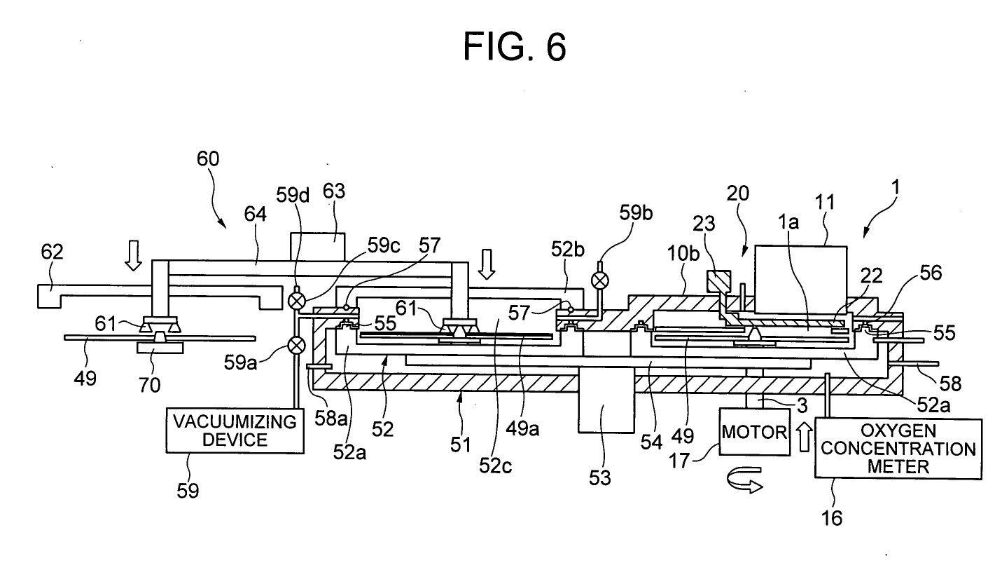

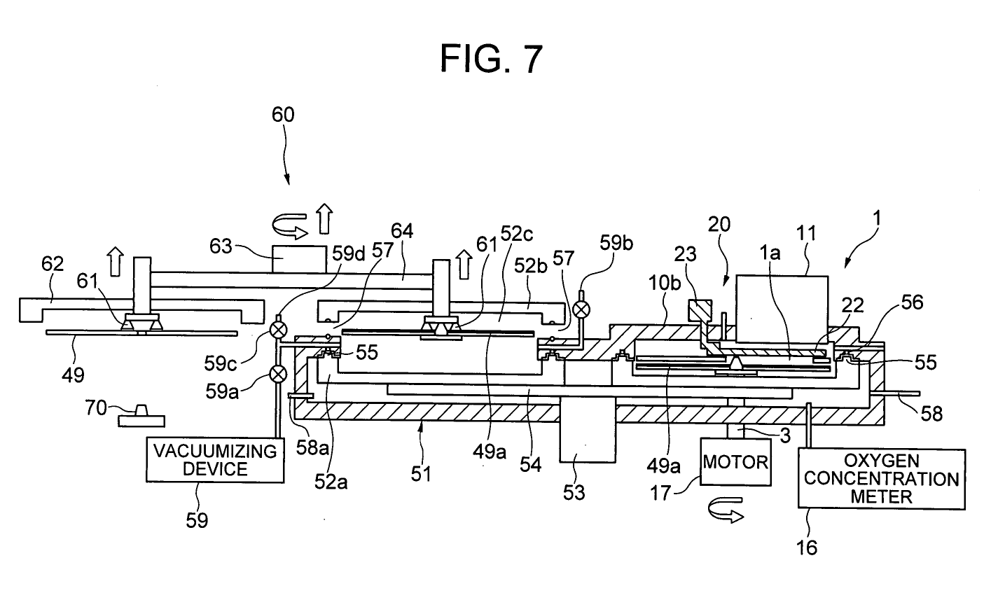

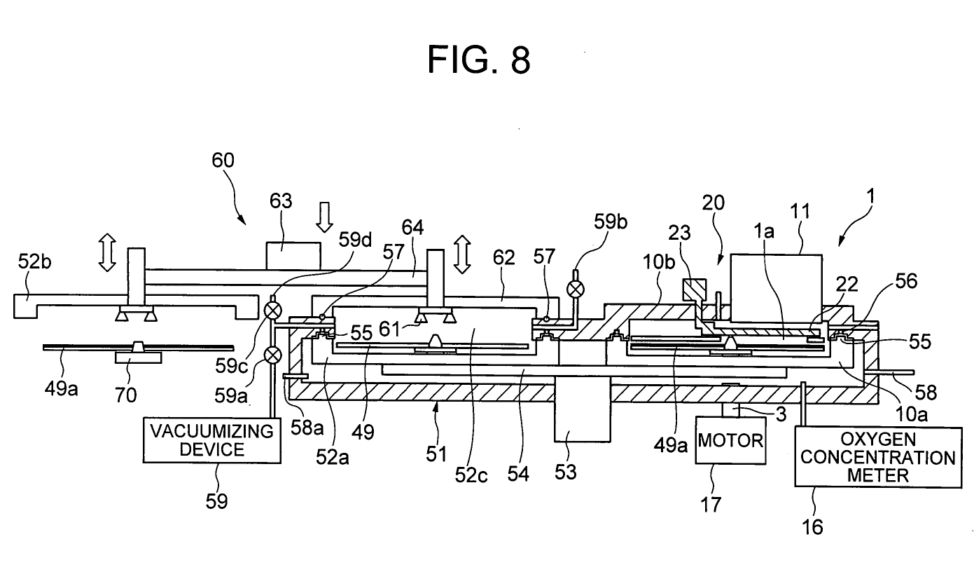

Next, as shown in FIG. 6, the shutter member 22 is opened by the shutter driving mechanism 20 (S24), the surface, formed with the lubricating layer on the light transmitting layer, of the on-rotating disc-shaped medium 49 is irradiated with the electron beams emitted from the electron beam irradiation unit 11 (S25). After the irradiation of the electron beams is effected for only a predetermined period of time as in FIG. 7, the shutter member 22 is closed by the shutter driving mechanism 20 as in FIG. 8 (S26), thereby finishing the irradiation of the electron beams upon the surface of the disc-shaped medium 49. This enables acquisition of the disc-shaped medium 49a including the lubricating layer fixed onto the surface of the light transmitting layer of the disc-shaped medium 49. This is considered such that the light transmitting layer is cured, and at the same time the reactive group of the lubricant is bound (cured) with reactive groups of the surface of the light transmitting layer and of other lubricant.

<Ejecting/Supplying of Disc-Shaped Medium>

In a state where the airtight closed space 52c is formed within the exchange chamber 52 as shown in FIG. 5, the airtight closed space 52c in the exchange chamber 52 accommodating the post-irradiation disc-shaped medium 49a inside is opened to the atmospheric air through an opening valve 59c and an opening port 59d as shown in FIG. 6 (S30).

Then, the disc carrying device 60 moves the adsorbing member 61 provided on the side of the carry rotational tray unit 52b downward as viewed in FIG. 6 through the rotary shaft 63 and the rotational unit 64, thereby absorbing the disc-shaped medium 49a (S31). Almost simultaneously with this, the adsorbing member 61 on the side of another carry rotational tray unit 62 adsorbs the pre-irradiation disc-shaped medium 49 formed with the light transmitting layer, etc. on its surface, which is accommodated in the external disc transferring/receiving unit 70 (S32).

Next, as illustrated in FIG. 7, the disc carrying device 60 raises the disc-shaped medium 49a from within the rotational tray unit 52a together with the absorbing member 61 and the carry rotational tray unit 52b by moving the rotary shaft 63 and the rotational unit 64 upward as viewed in FIG. 7, and simultaneously raises the disc-shaped medium 49 together with the absorbing member 61 and the carry rotational tray unit 62. Then, the rotational unit 64 rotates about the rotary shaft 63, whereby the carry rotational tray units 52b and 62 are replaced in their positions with each other (S33).

Next, as shown in FIG. 8, the disc carrying device 60 moves the rotary shaft 63 and the rotational unit 64 downward as viewed in FIG. 7, thereby setting the disc-shaped medium 49a into the rotational tray unit 52a of the exchange chamber 52 (S34). On the other hand, the disc-shaped medium 49a is transferred to the disc transferring/receiving unit 70 (S35), and the respective adsorbing members 61 stop adsorbing the disc-shaped mediums 49, 49a and move upward as viewed in FIG. 8. The disc-shaped medium 49a is ejected to the outside from the disc transferring/receiving unit 70 (S36).

Then, the airtight closed space 52c, which is formed again in the manner described above, within the exchange chamber 52 is depressurized by the vacuumizing device 59, and the nitrogen gas is introduced via the nitrogen gas introduction port 59b, wherein the replacement of the nitrogen gas is conducted beforehand (S37).

In the way described above, the disc-shaped medium 49a after being irradiated with the electron beams can be carried up to the disc transferring/receiving unit 70 from the exchange chamber 52, and at the same time the disc-shaped medium 49 before being irradiated with the electron beams can be carried up to the exchange chamber 52 from the disc transferring/receiving unit 70. Thus, the disc-shaped mediums 49, 49a can be exchanged by the single rotational operation of each of the rotary shaft 63 and the rotational unit 64.

Further, the exchange between the disc-shaped mediums 49, 49a can be efficiently executed during the irradiation of the electron beams by the electron beam irradiation apparatus 1 because of the airtight closed spaces 1a, 52c being independent of each other as shown in FIGS. 6 and 7.

Next, an exchanging operation of the disc-shaped medium between the exchange chamber 52 and the electron beam irradiation apparatus 1 will be explained. To be specific, as illustrated in FIG. 8, the rotational tray unit 52a of the exchange chamber 52 accommodates the pre-irradiation disc-shaped medium 49. In the electron beam irradiation apparatus 1, the rotation by the motor 17 is stopped (S38), the disc-shaped medium 49a that has finished being irradiated with the electron beams is housed in the rotational tray unit 10a, and, in this state, the rotary shaft 53 and the rotational unit 54 move downward as viewed in FIG. 8, whereby the rotational tray units 52a, 10a move downward to open the airtight closed spaces 52c, 10c. Note that the interior of the airtight closed space 52c has been replaced with the nitrogen gas atmosphere at that time, and hence there is no influence on other portions within the chamber 51.

Next, as shown in FIG. 9, the rotational unit 54 rotates about the rotary shaft 53 within the chamber 51, thereby exchanging the rotational tray units 52a, 10a in their positions with each other (S39). With this operation, the pre-irradiation disc-shaped medium 49 housed in the rotational tray unit 52a is moved into the electron beam irradiation apparatus 1 (S40), and, almost simultaneously with this, the disc-shaped medium 49a housed in the rotational tray unit 10a is moved into the exchange chamber 52 (S41).

In the way explained above, the disc-shaped mediums 49, 49a can be exchanged with each other between the exchange chamber 52 and the electron beam irradiation apparatus 1 by performing one rotational operation of each of the rotary shaft 53 and the rotational unit 54. Then, the rotary shaft 53 and the rotational unit 54 move upward as viewed in the Figure in order to move upward the rotational tray units 52a, 10a, whereby the airtight closed spaces 52c, 10a are again formed as shown in FIG. 5. Then, in the electron beam irradiation apparatus 1 the operation returns to step S22 described above, and in the exchange chamber 52 the operation goes back to step S30, thus enabling the same operations to be repeated.

Note that the rotary shaft 3 of the motor 17 is contrived to, when the rotary shaft 53 and the rotational unit 54 rotate, retreat downward from the rotational unit 54 and from the rotational tray unit 10a, thus permitting the rotational unit 54 to rotate.

As explained above, according to the manufacturing apparatus 50 in FIGS. 5 through 9, the disc-shaped medium 49 of which the surface is formed with the lubricating layer, etc. is rotated, and the on-rotating disc-shaped medium is irradiated with the electron beams whose acceleration voltage is as low as 20 kV through 100 kV. It is therefore possible to irradiate instantaneously the disc-shaped medium at a high efficiency with the electron beams exhibiting the greater energy than the ultraviolet rays have. This enables both of facilitation of curing and fixing the lubricating layer, etc. that is hard to be cured by the irradiation of the ultraviolet rays and the instantaneous formation of the lubricating layer, etc. As a result of improving the productivity for forming the lubricating layer, etc., this improvement can contribute to enhance the productivity of the disc-shaped medium.

Further, in the interior of the chamber 51 and in the disc carrying device 60, the two pieces of rotational tray units are exchanged with each other by the single rotational operation of each rotational tray unit in synchronization between one rotational tray unit and the other rotational tray unit, thereby ejecting the post-irradiation disc-shaped medium 49a and supplying the pre-irradiation disc-shaped medium 49. The disc-shaped mediums 49, 49a can be thus efficiently exchanged with each other, and hence the productivity is improved.

Still, because of using the electron beams of which the acceleration voltage is 20 Kv through 100 kV, the electron beam energy is efficiently applied to the lubricating layer and the resin layer existing over the thin range from the surface, and the electron beams do not affect the substrate existing thereunder.

Moreover, the irradiation of the electron beams can be conducted over the entire irradiated surface of the disc-shaped object so as to attain substantially the uniform distribution of the integrated irradiation dose of the electron beams in the radial direction of the rotated object 2, and the energy of the electron beams can be uniformly applied over the entire irradiated surface of the rotated object 2. With this contrivance, it is possible to substantially uniformly and instantaneously cure the lubricating layer and the resin layer over the entire surface at the high efficiency, whereby a quality and productivity of the disc-shaped object can be improved.

Further, the switchover control of the irradiation and the non-irradiation of the electron beams can be easily executed by using the shutter driving mechanism 20 and the shutter member 22, and there is no necessity of ON/OFF-controlling the power source 12 of the electron beam irradiation unit 11. Hence, the startup time of the electron beam irradiation unit 11 gets unnecessary, the disc-shaped mediums 49 are supplied one after another to the electron beam irradiation apparatus 1, and the repetitive irradiation of the electron beams can be consecutively executed at the high efficiency, thereby improving the productivity.

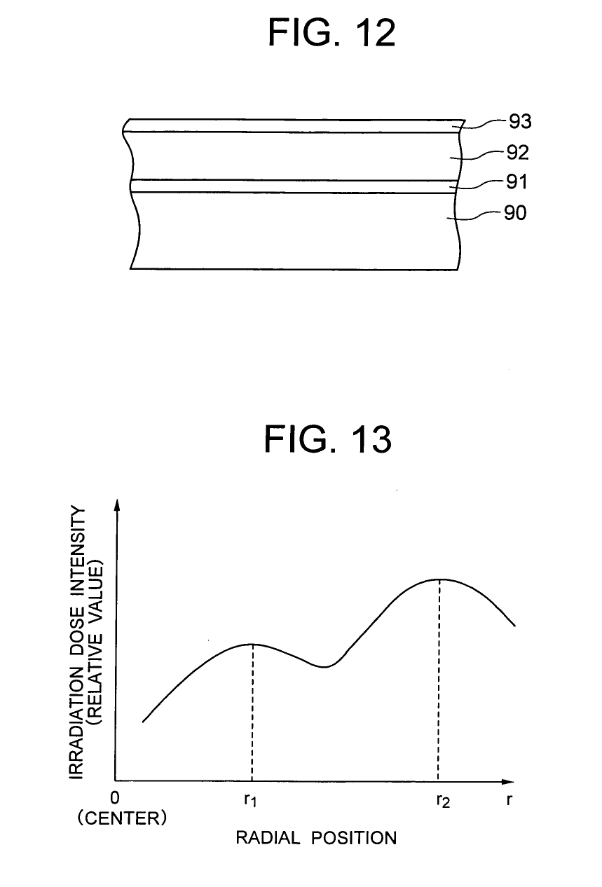

For example, the electron beam irradiation tubes 31, 32, 33 (FIGS. 2, 15), configuring the electron beam irradiation unit 11 of the electron beam irradiation apparatus 1, for irradiating the electron beams having the low acceleration voltage, are available on the market as offered by Ushio Electric Co., Ltd. The electron beam irradiation tube is capable of efficiently applying the electron beam energy to the lubricating layer/resin layer, etc. within a depthwise range that is on the order of 10 μm through 20 μm from the surface under the condition that the acceleration voltage is 50 kV, and a tube current is 0.6 mA per piece, and is capable of efficiently curing the layer instantaneously in less than 1 sec. For instance, the electron beam irradiation tube can simultaneously cure not only a lubricating layer 93 on the optical disc as shown in FIG. 12 but also even a portion, contiguous to the lubricating layer 93, of a light transmitting layer 92. Besides, for example, since the electron beams do not reach a substrate 90 existing under the lubricating layer 93 on the optical disc as illustrated in FIG. 12, and hence no damage is exerted on the substrate 90 composed of a resin material such as polycarbonate, etc., and there occurs none of adverse influence such as discoloration, deformation, deterioration and so forth.

Note that a window material for forming each of the irradiation windows 31b, 32b, 33b of the respective electron beam irradiation tubes 31 through 33 is preferably a silicon thin film having a thickness of approximately 3 μm, thereby making it possible to extract the electron beams accelerated at the acceleration voltage that is as low as 100 kV or under, which can not be extracted by the conventional irradiation window.

Moreover, throughout the present specification, the term [rotational] implies not a simple consecutive rotation of the disc-shaped object in one direction (or in the direction opposite thereto) as in the rotation but a turn in a way that changes its position so as to turn by a predetermined amount in one direction or in the opposite direction and then stop.

As discussed above, the present invention has been described by way of the embodiments but is not limited to those embodiments, and a variety of modifications can be made within the range of the technical ideas of the present invention. For example, in the apparatus for manufacturing the disc-shaped medium according to the present embodiment, the exemplification is that the light transmitting layer and the lubricating layer that are composed of the aforementioned materials are formed by curing in the vicinity of the surface of the disc-shaped medium such as an optical disc, etc., however, the present invention is not limited to this construction and may also be, as a matter of course, applied to the curing of a resin layer, etc. other than the lubricating layer. For instance, the present invention may be applied to forming, in FIG. 12, only the light transmitting layer 92 under the lubricating layer 93, wherein the layer can be instantaneously cured. This is efficient and can contribute to the improvement of the productivity.

Moreover, a variety of disc shapes may be taken for the disc-shaped object that can be irradiated with the electron beams by the electron beam irradiation apparatus 1. Further, the disc-shaped medium such as the optical disc, etc. has been exemplified as the disc-shaped object that can be manufactured by the manufacturing apparatus 50, however, the present invention can be, as a matter of course, applied to a case of forming a variety of resin layers on the disc-shaped object other than the medium.

Still further, in the electron beam irradiation apparatus in FIG. 1 and in the manufacturing apparatus in FIGS. 5 through 9, it is preferable that the acceleration voltage, etc. of the electron beam irradiation tube of the electron beam irradiation unit 11 is determined in consideration of the layer thickness on the surface defined as the target irradiated with the electron beams. Yet further, the number of electron beam irradiation tubes configuring the electron beam irradiation unit 11 can be properly increased or decreased corresponding to a size and an area of the irradiation target surface.

Furthermore, the gas to be replaced with the atmospheres within the chamber and within the electron beam irradiation apparatus is not limited to the nitrogen gas, wherein an inert gas such as an argon gas, a helium gas, CO₂, etc. is available, and a mixture gas of these two or more types of gases is also available.

Further, in FIGS. 1, 2 and 14, the number of the electron beam irradiation tubes is set to 2 but may also be set equal to or larger than 3. In this case, the configuration is such that the irradiation beam intensity of the electron beams becomes larger toward the side of the outer peripheral surface from the side of the inner peripheral surface in the radial direction on the irradiated surface of the disc-shaped object.

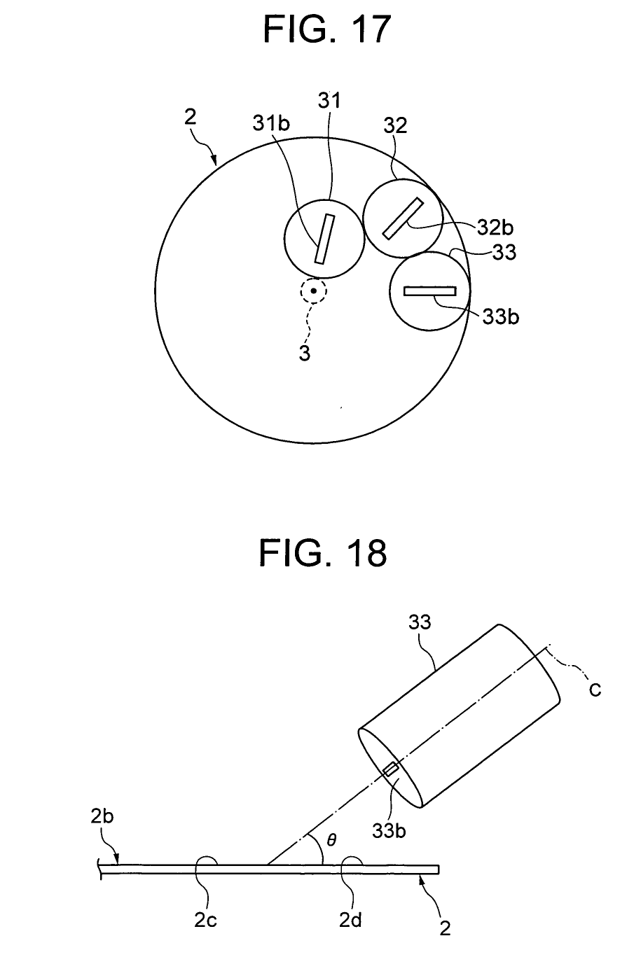

Moreover, the plurality of electron beam irradiation tubes may be arranged, as shown in FIG. 2, in the same radial direction (on the straight line extending in the radial direction) on the disc-shaped object. As shown in FIG. 16, however, the two electron beam irradiation tubes 31, 32 may also be arranged so as to be substantially vicinal to each other in the different radial directions (on a plurality of straight lines differently extending in the radial directions) on the disc-shaped object 2. Moreover, as shown in FIG. 17, the three electron beam irradiation tubes 31, 32, 33 may also be so arranged as to be substantially neighboring to each other in different radial directions (on a plurality of straight lines differently extending in the radial directions) on the disc-shaped object 2.

Still further, in FIGS. 2, 16 and 17, the respective irradiation windows 31b through 33b are arranged along the straight lines in the radial directions extending radially from the center of the rotary shaft 3 but are not limited to this arrangement and may also be so arranged as to be inclined at a predetermined angle to those straight lines.

### INDUSTRIAL APPLICABILITY

According to the present invention, it is possible to provide the electron beam irradiation apparatus and the electron beam irradiation method each capable of easily curing the material that is hard to be cured by the irradiation of the ultraviolet rays and of substantially uniformizing the integrated irradiation dose of the electron beams over the entire irradiated surface.

Moreover, it is possible to provide the disc-shaped object manufacturing apparatus and the disc-shaped object manufacturing method each capable of substantially uniformizing the integrated irradiation dose of the electron beams over the entire irradiated surface and efficiently forming, on the disc-shaped object, the lubricating layer, the resin layer, etc. composed of the material that is hard to be cured by the irradiation of the ultraviolet rays.

1. An electron beam irradiation apparatus comprising:
a rotary driving unit for rotationally driving a disc-shaped object;
a shield container for rotatably accommodating the disc-shaped object; and
an electron beam irradiation unit provided in said shield container so that a face to be irradiated on the surface of the disc-shaped object is irradiated with electron beams,
wherein when the face to be irradiated is irradiated with the electron beams emitted from said electron beam irradiation unit during rotations of the disc-shaped object, an irradiation beam intensity of the electron beams is set larger on the side of an outer peripheral surface in a radial direction of the disc-shaped object than on the side of an inner peripheral surface.

2. An electron beam irradiation apparatus according to claim 1, wherein an acceleration voltage of said electron beam irradiation unit is 20 kV through 100 kV.

3. An electron beam irradiation apparatus according to claim 1, wherein said electron beam irradiation unit includes a plurality of electron beam irradiation tubes arranged in the radial direction.

4. An electron beam irradiation apparatus according to claim 3, wherein each of current values of said plurality of electron beam irradiation tubes is set so that the current value of said electron beam irradiation tube disposed on the side of the outer peripheral surface is larger than the current value of said electron beam irradiation tube disposed on the side of the inner peripheral surface.

5. An electron beam irradiation apparatus according to claim 3, wherein said plurality of electron beam irradiation tubes respectively have irradiation windows through which the electron beams are irradiated toward the outside, and are arranged so that a distance from the face to be irradiated to said irradiation window is shorter in said electron beam irradiation tube on the side of the outer peripheral surface than a distance in said electron beam irradiation tube on the side of the inner peripheral surface.

6. An electron beam irradiation apparatus according to claim 3, wherein at least one of said plurality of electron beam irradiation tubes is disposed so that said irradiation window thereof is inclined close to the side of the outer peripheral surface of the face to be irradiated.

7. An electron beam irradiation apparatus according to claim 3, wherein said plurality of electron beam irradiation tubes are arranged substantially in the same direction along the radial direction.

8. An electron beam irradiation apparatus according to claim 3, wherein said plurality of electron beam irradiation tubes are arranged in different directions in the radial direction.

9. An electron beam irradiation apparatus according to claim 1, wherein said electron beam irradiation unit includes an electron beam irradiation tube having an irradiation window through which the electron beams are irradiated to the outside, and
said electron beam irradiation tube is disposed so that said irradiation window thereof is inclined close to the side of the outer peripheral surface of the face to be irradiated.

10. An electron beam irradiation apparatus according to claim 1, wherein an interior of said shield container is set in an atmosphere of an inert gas, and
said shield container is provided with a gas introduction port and a gas discharge port through which the inert gas flows in the vicinity said irradiation window.

11. An electron beam irradiation apparatus according to claim 10, wherein a temperature sensor is provided in the vicinity of said electron beam irradiation unit, and
a flow rate of the inert gas is adjusted based on a temperature measured by said temperature sensor.

12. An electron beam irradiation apparatus according to claim 1, wherein an oxygen concentration meter for measuring an oxygen concentration within said shield container, is provided.

13. An electron beam irradiation apparatus according to claim 1, wherein a vacuumizing device for depressurizing the interior of said shield container is provided.

14. An electron beam irradiation apparatus according to claim 1, wherein said shield container is openable/closable and composed of a metallic material, and has a shield structure for shielding the electron beams emitted from said irradiation window.

15. An electron beam irradiation apparatus according to claim 1, further comprising: a shutter member disposed between said electron beam irradiation unit and the irradiated surface, and movable between an opening position for opening to permit transmission of the electron beams and a closing position for closing to block the electron beams; and
a shutter driving mechanism for moving said shutter member so as to effect switchover to the irradiation and non-irradiation of the electron beams during rotations of said disc-shaped object.

16. An electron beam irradiation apparatus according to claim 15, wherein said shutter member is constructed to open and close at a higher speed than a peripheral speed on the outer periphery of said disc-shaped object.

17. An electron beam irradiation method comprising the steps of:
rotationally driving a disc-shaped object; and
irradiating a face to be irradiated of the on-rotating disc-shaped object with the electron beams emitted from an electron beam irradiation unit so that an irradiation beam intensity of the electron beams is set larger on the side of an outer peripheral surface in a radial direction of the disc-shaped object than on the side of an inner peripheral surface.

18. An electron beam irradiation method according to claim 17, wherein an acceleration voltage of said electron beam irradiation unit is 20 kV through 100 kV.

19. An electron beam irradiation method according to claim 17, further comprising the step of setting current values of a plurality of electron beam irradiation tubes arranged in the radial direction serving as said electron beam irradiation so that the current value of said electron beam irradiation tube disposed on the side of the outer peripheral surface is larger than the current value of said electron beam irradiation tube disposed on the side of the inner peripheral surface.

20. An electron beam irradiation method according to claim 17, further comprising the step of setting a distance from the irradiated surface to each of said electron beam irradiation windows of said plurality of electron beam irradiation tubes arranged in the radial direction serving as said electron beam irradiation unit so that the distance in said electron beam irradiation tube on the side of the outer peripheral surface is shorter than the distance in said electron beam irradiation tube on the side of the inner peripheral surface.

21. An electron beam irradiation method according to claim 19, further comprising the step of inclining at least one of said plurality of electron beam irradiation tubes so that said irradiation window thereof gets close to the side of the outer peripheral surface of the face to be irradiated.

22. An electron beam irradiation method according to claim 17, further comprising the step of inclining said electron beam irradiation tube arranged as said electron beam irradiation unit so that said irradiation windows thereof get close to the side of the outer peripheral surface of the irradiated surface.

23. An electron beam irradiation method according to claim 17, further comprising the steps of:
rotatable accommodating said disc-shaped object:
in an air-tightly closable shield container and
replacing an interior of said shield container with an inert gas atmosphere by introducing an inert gas into the interior of said shield container.

24. An electron beam irradiation method according claim 23, further comprising the step of flowing the inert gas through the vicinity of said irradiation window of said electron beam irradiation unit toward a gas discharge port from a gas introduction port, thereby cooling off said irradiation window.

25. An apparatus for manufacturing a disc-shaped object, comprising an electron beam irradiation apparatus according to claim 1,
further comprising at least one of a resin layer and a surface layer formed on said disc-shaped object cured by the irradiation of the electron beams.

26. A method of manufacturing a disc-shaped object, involving the use of an electron beam irradiation apparatus according to claim 17,
further comprising the step of curing at least one of a resin layer and a surface layer formed on said disc-shaped object by the irradiation of the electron beams.
